(19) United States
(12) Patent Application Publication
(10) Pub. No.: US 2024/0021291 A1
(43) Pub. Date: Jan. 18, 2024

(54) System and Method of Facilitating Digital Therapy for Long-Term Neuropsychological Rehabilitation

(71) Applicant: ABAStroke Sp. z o.o., Krakow (PL)

(72) Inventor: Michal Rys, Krakow (PL)

(21) Appl. No.: 17/865,250

(22) Filed: Jul. 14, 2022

(51) Int. Cl. G16H 20/70

(52) U.S. Cl. CPC G16H 20/70; G06F 3/04842

(57) ABSTRACT

A system and a method of facilitating digital therapy are used to provide long-term neuropsychological rehabilitation for patients with physical and cognitive disabilities. A user account is prompted to initiate a plurality of rehabilitation tasks with a corresponding personal computing (PC) device. The user account is then prompted to complete a specific task with the corresponding PC device. At least one specific hint for the specific task is outputted, if the specific task is improperly completed by the user account. If the specific hint is outputted, and until the specific task is properly completed by the user account, the previous step is repeated. If the specific task is properly completed by the user account, at least one reinforcement notification for the specific task is outputted. Then, a plurality of iterations is executed for the previous steps, until each rehabilitation task is properly completed by the user account.

### FIELD OF THE INVENTION

The present invention generally relates to medical systems and home-based treatments. More specifically, the present invention provides a digital system and method for treatment of post-stroke cognitive deficits which can be performed at home.

### BACKGROUND OF THE INVENTION

Nowadays, a stroke is one of the main causes of disability in the western world, with 30-35% of people struggling with cognitive impairments and memory loss after having a stroke and their living standards dropping dramatically. Unfortunately, access to free, long-term rehabilitation of cognitive functions is limited in various countries. The healthcare systems in many European Union (EU) countries can provide reasonably good health service but only at the beginning, during hospitalization, and during the primary rehabilitation process (usually one to two months available mostly to patients with associated mobility impairments). However, the huge gap in care occurs when patients return home. Lack of neuropsychologists and speech therapists makes further rehabilitation very limited to private treatment. Moreover, post-stroke care often focuses exclusively upon the physical impact of the stroke. There is less focus on memory and cognition even though stroke survivors with cognitive impairments often report feelings of abandonment when they leave the hospital. Daily rehabilitation for at least 30 minutes is often recommended by experts and only a few can afford to hire specialists for daily care. Furthermore, the growing aging population has increased the number of stroke cases and the problem will be magnified in the coming years. Urgent action is needed to provide means of at-home neuropsychological rehabilitation.

An objective of the present invention is to provide a system and a method that facilitate long-term neuropsychological rehabilitation for stroke patients. The present invention is dedicated to those who return home after a stay in a neurology ward or a rehabilitation clinic and lack the means for daily rehabilitation of cognitive functions. The present invention allows post-stroke patients to regain their quality of life through a recovery process comprising regular exercises via a digital system designed by neuropsychologists, neurologists, speech therapists, and developed by machine learning experts. The system and the method of digital rehabilitation of the present invention focuses on five main domains: attention control, working memory, executive functions, perception, and language. The present invention is aimed at therapy at home using a personal computing device and considers other deficits such as physical disabilities. Additional features and benefits of the present invention are further discussed in the sections below.

### SUMMARY OF THE INVENTION

The present invention provides a system and a method that are specifically tailored to treat patients with physical and cognitive disabilities, which are very common after a stroke incident. The treatment method used by the present invention is based on three principles: Applied Behavior Analysis (ABA), adapting to patients' needs, and facilitating home rehabilitation via a Personal Computing (PC) device. The foundations of the rehabilitation method of the present invention are based on ABA methods, which are the leading, research-based methodology proven to bring about meaningful change in behavior in individuals with cognitive disabilities. ABA treatment for post-stroke patients is one of the most effective ways for individuals with recognized behavior, mood, or emotional disorders. Further, the system of the present invention is highly adaptive and enables the patient to work on rehabilitation independently from home using an appropriate PC device.

Because of its adaptive capabilities, the system of the present invention allows the patients to receive the treatment independently or with minimal assistance provided by a family member or caretaker. Stroke patients are able to understand things faster, make decisions with less hesitation, and their confidence increases by utilizing the present invention. The cognitive functioning of the patient improves by using the present invention, which is a vital part for a better quality of life. Through regular exercises, patients make real progress on their rehabilitation which also leads to higher motivation for further rehabilitation.

### BRIEF DESCRIPTION OF THE DRAWINGS

FIG. 1 is a block diagram showing the system of the present invention.

FIG. 2 is a block diagram showing a user interface (UI) of the present invention, wherein the UI shows at least one task instruction.

FIG. 3 is a block diagram showing a user interface (UI) of the present invention, wherein the UI shows a plurality of visual elements, and wherein the plurality of visual elements includes a unique element and a plurality of identical elements.

FIG. 4 is a block diagram showing a user interface (UI) of the present invention, wherein the UI shows the selected unique element graphically enlarged and bordered, and wherein the plurality of identical elements is shown graphically reduced in opacity.

FIG. 5 is a block diagram showing a user interface (UI) of the present invention, wherein the UI shows a selected identical element of the plurality of identical elements graphically reduced in opacity.

FIG. 6 is a block diagram showing a user interface (UI) of the present invention, wherein the UI shows a selected identical element of the plurality of identical elements graphically removed.

FIG. 7 is a block diagram showing a user interface (UI) of the present invention, wherein the UI shows a plurality of new visual elements, and wherein the plurality of new visual elements includes a new unique element and a plurality of new identical elements.

FIG. 8 is a flowchart illustrating the overall process for the method of the present invention.

FIG. 9 is a flowchart illustrating the subprocess of providing at least one task instruction for the specific task.

FIG. 10 is a flowchart illustrating the subprocess of providing a plurality of visual elements for the specific task, wherein the plurality of visual elements includes a unique element and a plurality of identical elements.

FIG. 11 is a flowchart illustrating the subprocess of graphically reducing the opacity of a specific element, if the specific element is not the unique element.

FIG. 12 is a flowchart illustrating the subprocess of graphically removing a specific element, if the specific element is not the unique element.

FIG. 13 is a flowchart illustrating the subprocess of providing a new plurality of visual elements for a specific task, wherein the plurality of visual elements includes a new unique element and a plurality of new identical elements, if the specific element is not the unique element.

FIG. 14 is a flowchart illustrating the subprocess of graphically enlarging a specific element, if the specific element is the unique element.

FIG. 15 is a flowchart illustrating the subprocess of graphically bordering a specific element, if the specific element is the unique element.

FIG. 16 is a flowchart illustrating the subprocess of graphically reducing the opacity of the plurality of identical elements, if the specific element is the unique element.

FIG. 17 is a flowchart illustrating the subprocess of providing at least one task reinforcement message for the specific task.

FIG. 18 is a flowchart illustrating the subprocess of providing a task completion timer for the specific task.

FIG. 19 is a flowchart illustrating the subprocess of tracking a completion progression of the plurality of rehabilitation tasks.

FIG. 20 is a flowchart illustrating the subprocess of providing a patient rehabilitation history.

FIG. 21 is a flowchart illustrating the subprocess of accessing the patient rehabilitation history.

FIG. 22 is a flowchart illustrating the subprocess of providing a plurality of new rehabilitation tasks.

### DETAILED DESCRIPTIONS OF THE INVENTION

All illustrations of the drawings are for the purpose of describing selected versions of the present invention and are not intended to limit the scope of the present invention.

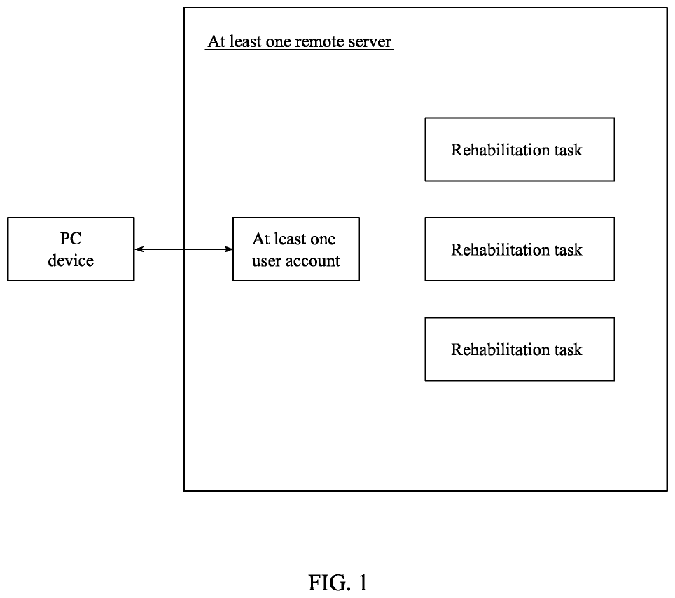

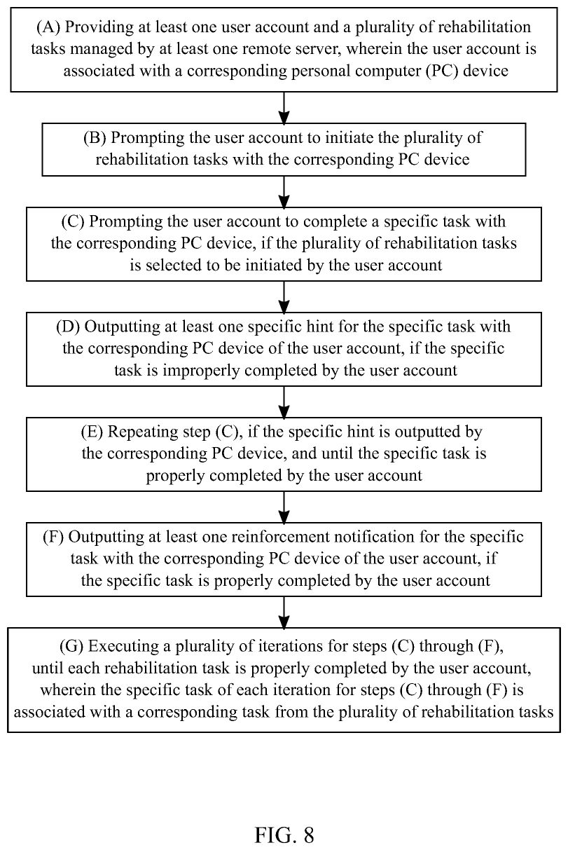

The present invention is a system and a method of facilitating digital therapy for long-term neuropsychological rehabilitation for patients with physical and cognitive disabilities, which are very common after a stroke incident. As can be seen in FIGS. 1 and 8, to do so, the system of the present invention may include at least one remote server that manages at least one user account and a plurality of rehabilitation tasks (Step A). The remote server is a central communication hub that manages the treatment programs provided to the user account for the long-term rehabilitation of the user. Through the user account the user is able to access the treatment necessary to receive long-term neuropsychological rehabilitation. Further, the user account is associated with a corresponding personal computing (PC) device. The corresponding PC device allows the user to interact with the treatment system of the present invention and can be, but is not limited to, a laptop, a desktop, a tablet computer, or a mobile computerized phone (i.e., a smartphone). Furthermore, the treatment programs may be provided as a specific software application compatible with the corresponding PC device, such as a mobile application (app), or as a software application that can be accessed through other third-party applications, such as a website that can be accessed through a web server.

An overall process for the method of the present invention allows post-stroke patients to regain their quality of life through a recovery process that includes regular exercises via various programs designed by neuropsychologists, neurologists, speech therapists, and developed by machine learning experts. As can be seen in FIG. 8, the overall process begins by prompting the user account to initiate the plurality of rehabilitation tasks with the corresponding PC device (Step B). The initiation of the plurality of rehabilitation tasks can be performed by the user engaging with the user interface of the corresponding PC device, such as clicking on the corresponding command to start a rehabilitation task of the plurality of rehabilitation tasks. Then, the user account is prompted to complete a specific task with the corresponding PC device, if the plurality of rehabilitation tasks is selected to be initiated by the user account (Step C). The specific task can be any of the plurality of rehabilitation tasks that may be launched in a specific order or randomly depending on the treatment programs appropriate to the user. Then, at least one specific hint for the specific task is outputted with the corresponding PC device of the user account, if the specific task is improperly completed by the user account (Step D). Due to the user suffering from possible physical and cognitive deficiencies, the specific hint serves as an aid to help the user solve the specific task if the user finds trouble solving the specific task. If the specific hint is outputted by the corresponding PC device, and until the specific task is properly completed by the user account, Step C is repeated (Step E). The repetition of the specific task helps the user improve the user's deficiencies with the help of the specific hint until the user is able to successfully complete the specific task. If the specific task is properly completed by the user account, at least one reinforcement notification for the specific task is outputted with the corresponding PC device of the user account (Step F). The reinforcement notification for the specific task serves as positive reinforcement for the user once the specific task is successfully completed. Then, a plurality of iterations for Steps C through F is executed, until each rehabilitation task is properly completed by the user account (Step G). The specific task of each iteration for Steps C through F is associated with a corresponding task from the plurality of rehabilitation tasks. The number of rehabilitation tasks depends on the treatment programs developed for the user, as well as the type and level of difficulty of the rehabilitation tasks.

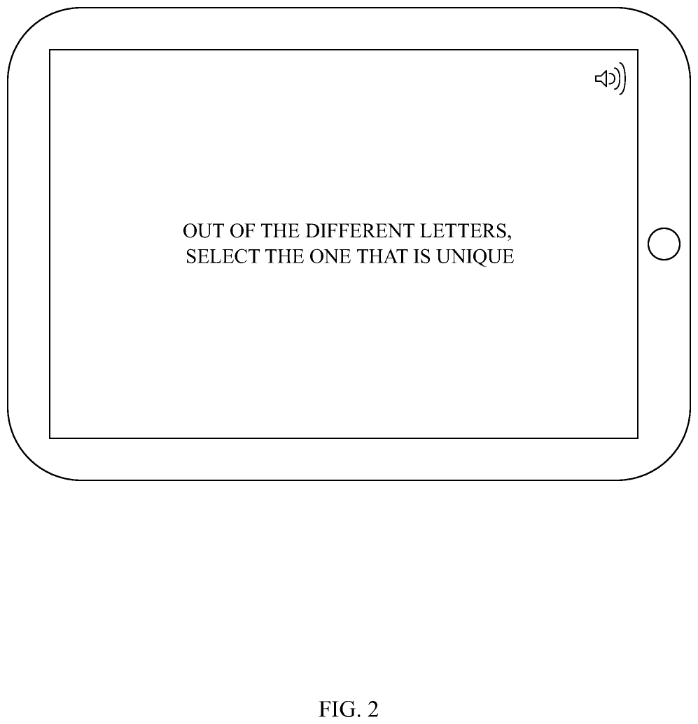

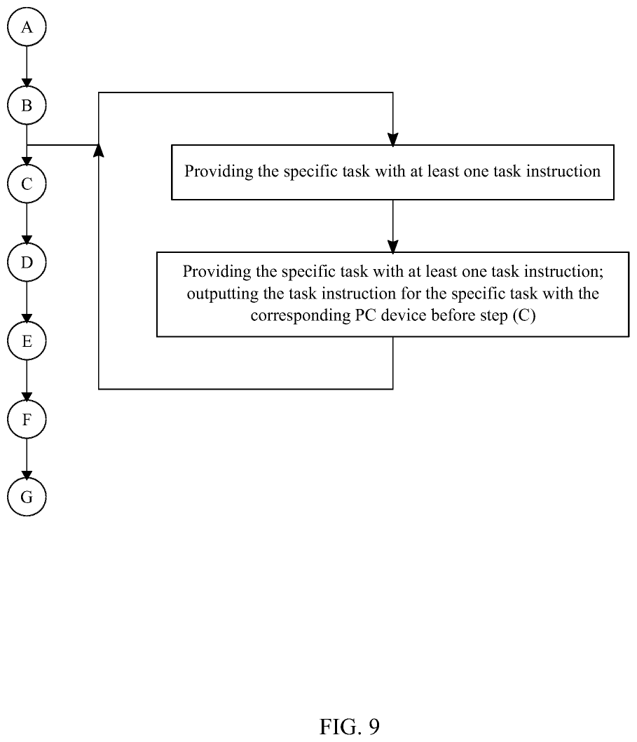

Before the user starts working on the specific task from the plurality of rehabilitation tasks, the user is presented with instructions on how to successfully complete the specific task. As can be seen in FIGS. 2 and 9, to do so, the system of the present invention further provides the specific task with at least one task instruction. The task instruction includes various information regarding the specific task that directs the user on the correct way to complete the specific task. For example, the task instruction may include written instructions or audio instructions that instructs the user what steps that the user must follow to successfully complete the specific task. The subprocess of providing task instructions includes the step of outputting the task instruction for the specific task with the corresponding PC device before Step C. This way, the user has the necessary information to successfully complete the specific task before starting to work on the specific task.

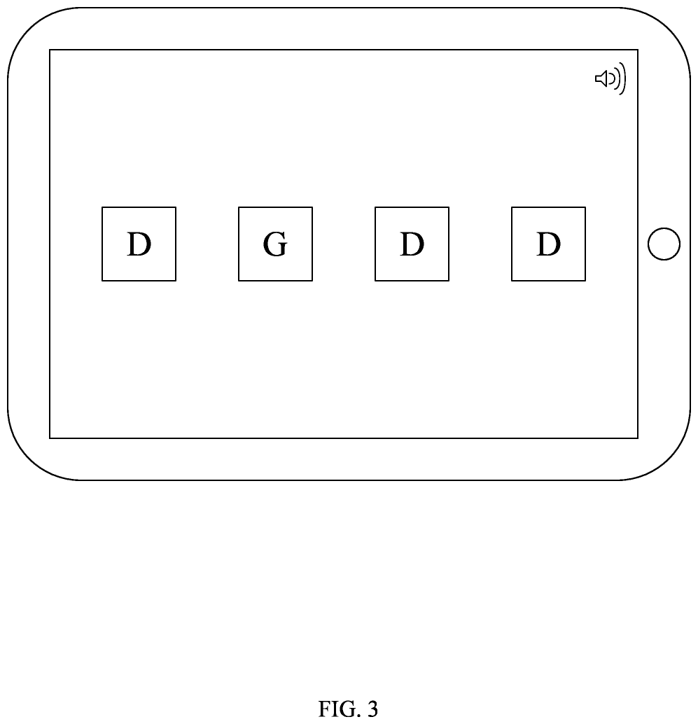

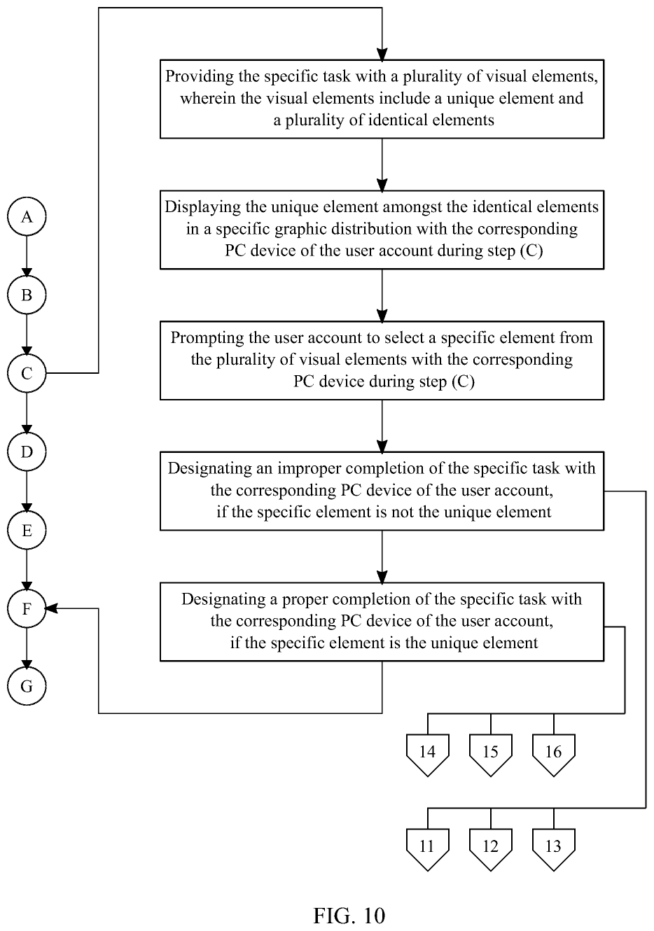

The present invention enables the user to receive long-term neuropsychological rehabilitation by performing different rehabilitation tasks that engage various cognitive functions, such as recognizing patterns of different visual objects. As can be seen in FIGS. 3 and 10, to do so, the system of the present invention may further include a plurality of visual elements. The visual elements are graphical elements such as letters, numbers, pictures, or a combination thereof. The visual elements are provided in different patterns and arrangements that include a unique element and a plurality of identical elements. In other words, the visual elements are provided with a group of identical elements (same visual element repeated an nth number of times) and a single unique element. The goal of the specific task is then for the user to identify and select the unique element that is positioned among the plurality of identical elements. The subprocess of setting up the visual elements for a specific task starts by displaying the unique element amongst the identical elements in a specific graphic distribution with the corresponding PC device of the user account during Step C. For example, the specific graphic distribution can include the unique element positioned in between multiple identical elements to confuse the user. Alternatively, the unique element can be position can be positioned so that the unique element can be more easily differentiated from the identical elements, such as positioning the unique element separate from the identical elements. Then, the user account is prompted to select a specific element from the plurality of visual elements with the corresponding PC device during Step C. In other words, the user can select any of the visual elements regardless of the specific element being the unique element or an identical element. Moreover, if the specific element selected by user account is not the unique element, an improper completion of the specific task is designated with the corresponding PC device of the user account. In other words, if the user selects an identical element from the plurality of identical elements, the specific task has not been properly completed by the user. Alternatively, if the specific element selected by the user account is the unique element, a proper completion of the specific task is designated with the corresponding PC device of the user account. In other words, the user must select the unique element to properly completed the specific task.

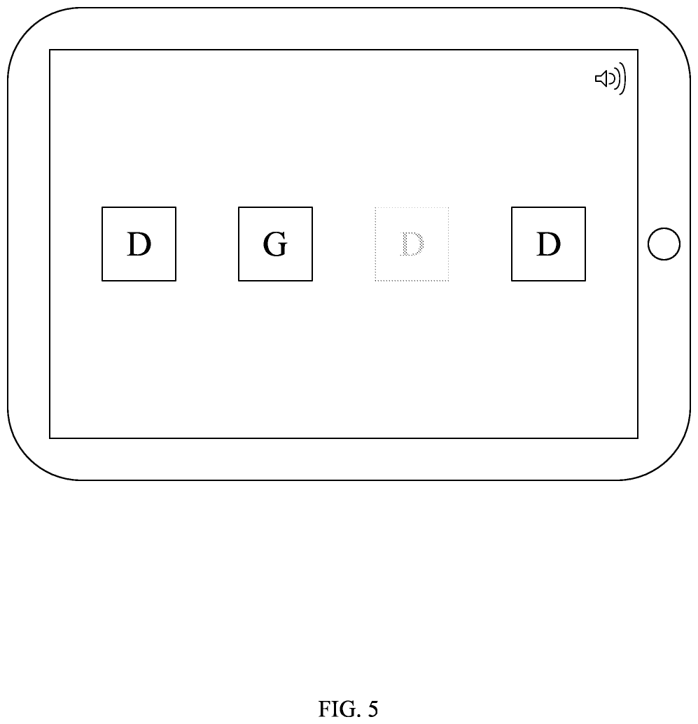

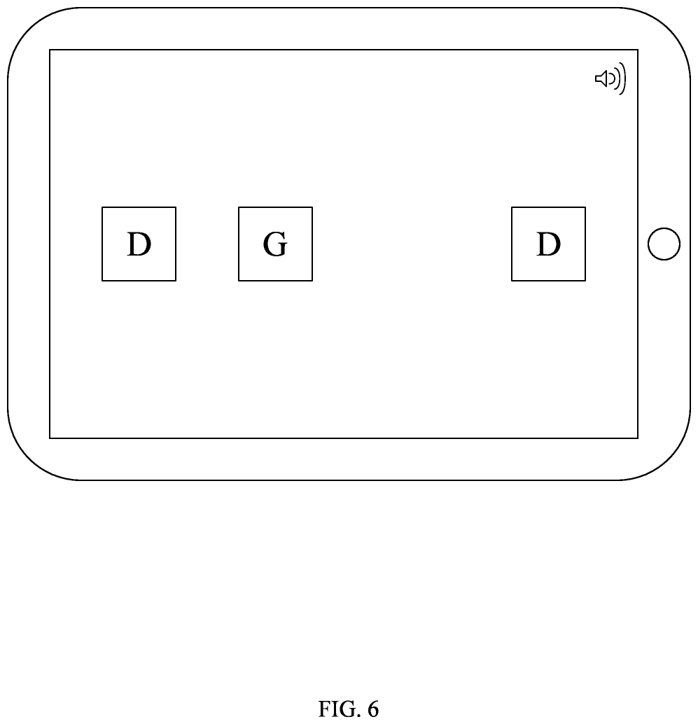

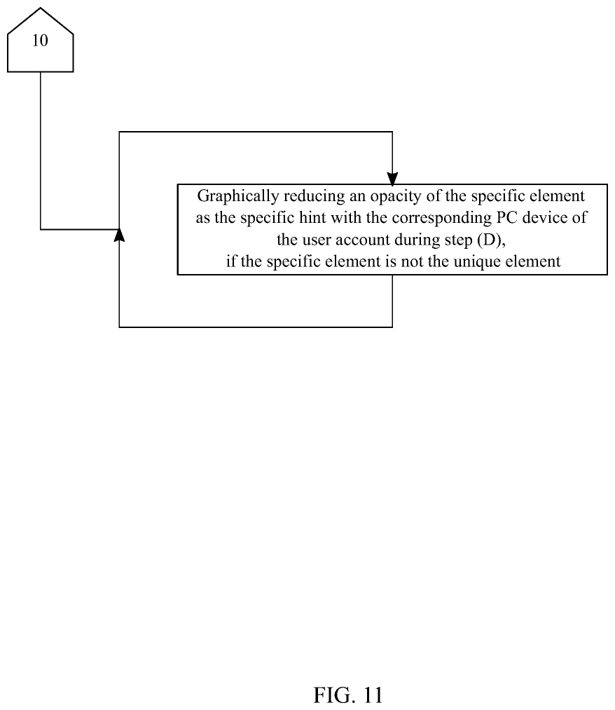

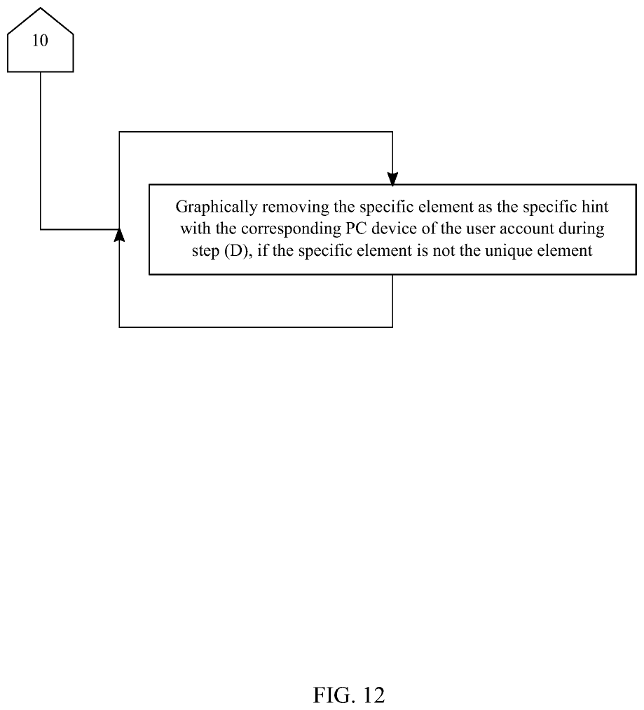

As previously discussed, the user is provided with feedback regarding the results of the specific task completion. The feedback can be communicated through different means to accommodate the user's needs. For example, the feedback includes the specific hint and can be, but is not limited to, a visual or audible hint. This way, the specific hint can provide different information depending on the result of the specific task completion by the user. As can be seen in FIGS. 5 and 11, if the specific element is not the unique element, an opacity of the specific element is graphically reduced as the specific hint with the corresponding PC device of the user account during Step D. In other words, the selected specific element is visually faded to indicate that the chosen specific element is not the unique element. In addition to fading the chosen specific element, the user is given additional help so that the user can perform better in the next rehabilitation task. Alternatively, if the specific element is not the unique element, the specific element is graphically removed as the specific hint with the corresponding PC device of the user account during the Step D, which can be seen in FIGS. 6 and 12. In other words, the possible choices are reduced to facilitate the next rehabilitation user for the user.

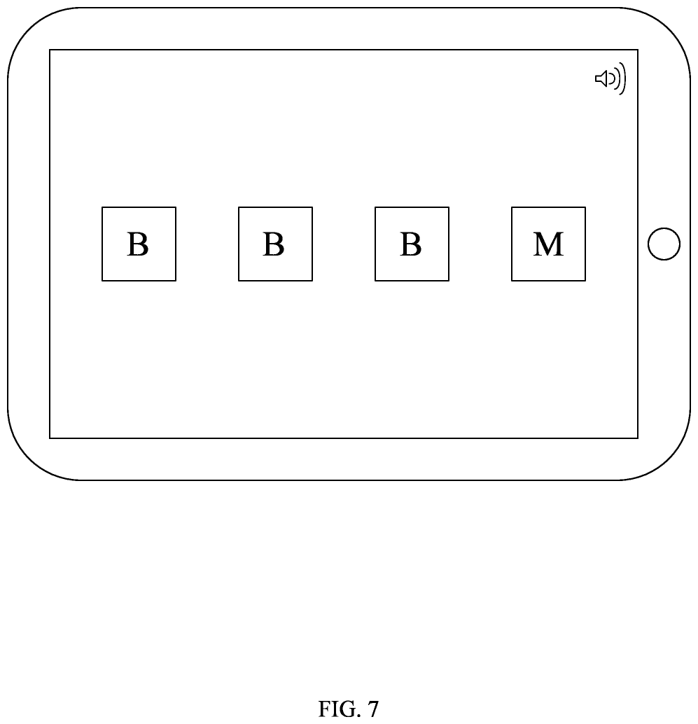

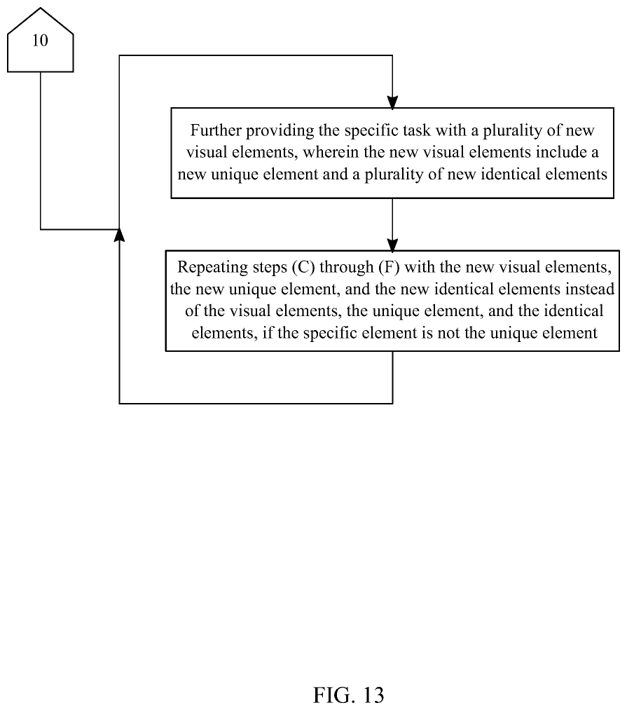

Alternatively, if the user fails to choose the unique element, the user can be presented with a new rehabilitation task. After the improper completion of the specific task, a variation of the specific task is provided by the system of the present invention. As can be seen in FIGS. 7 and 13, the system of the present invention further provides the specific task with a plurality of new visual elements. The plurality of new visual elements includes a new set of visual elements that may include, but is not limited to, new set of letters, numbers, pictures, or a combination thereof. In addition, the new visual elements include a new unique element and a plurality of new identical elements. The subprocess of providing a variation of the specific task begins by repeating Steps C through F with the new visual elements, the new unique element, and the new identical elements instead of the visual elements, the unique element, and the identical elements, if the specific element is not the unique element. This way, the user is further challenged to try again by performing a variation of the specific task.

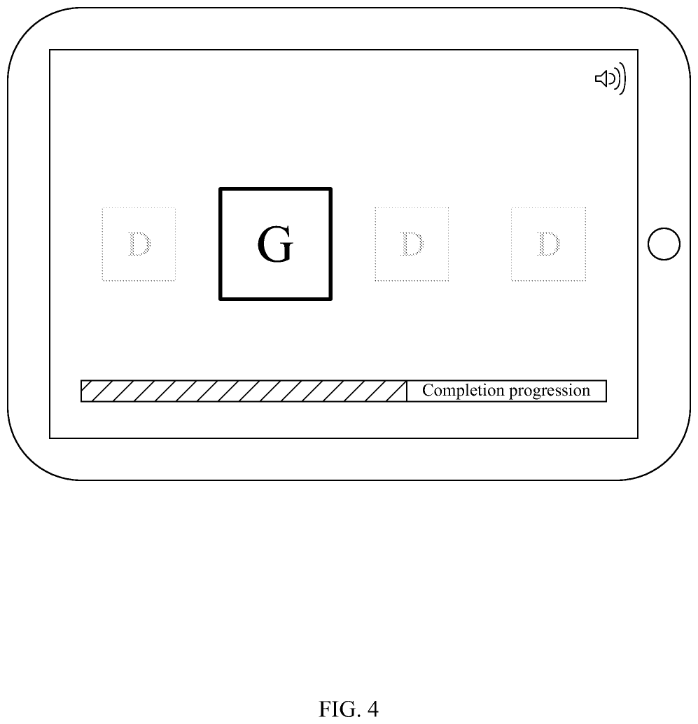

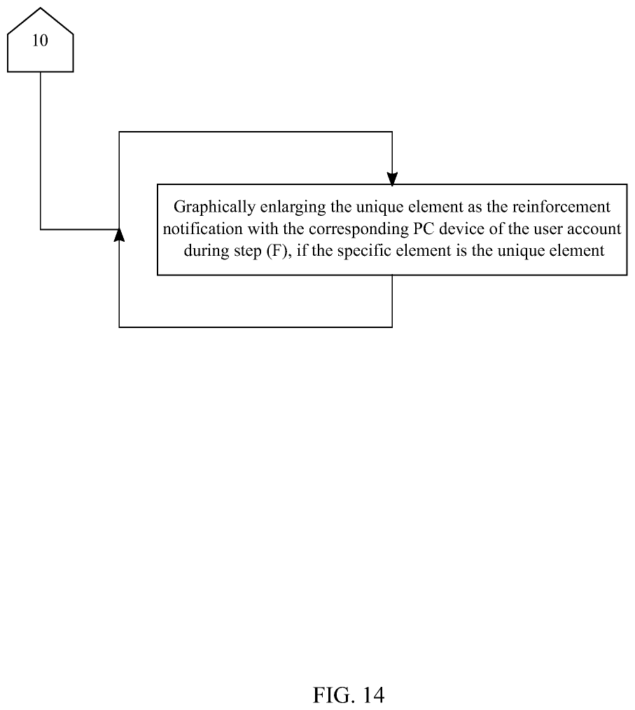

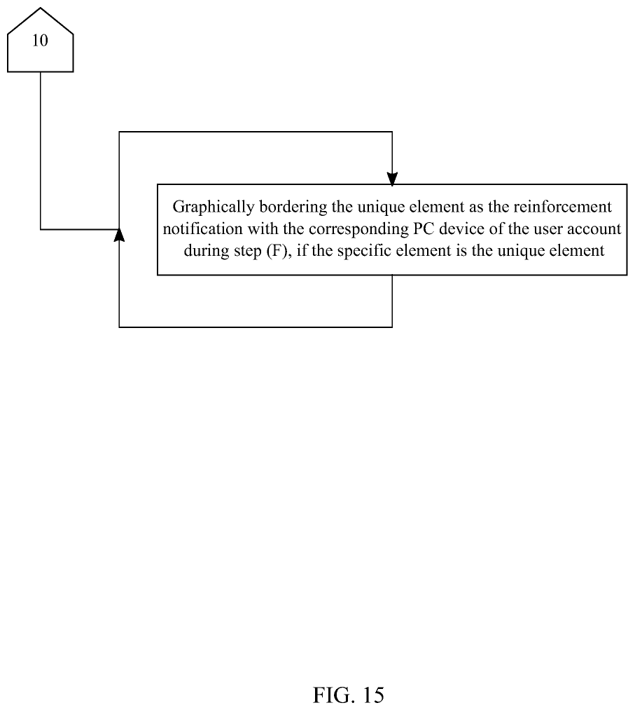

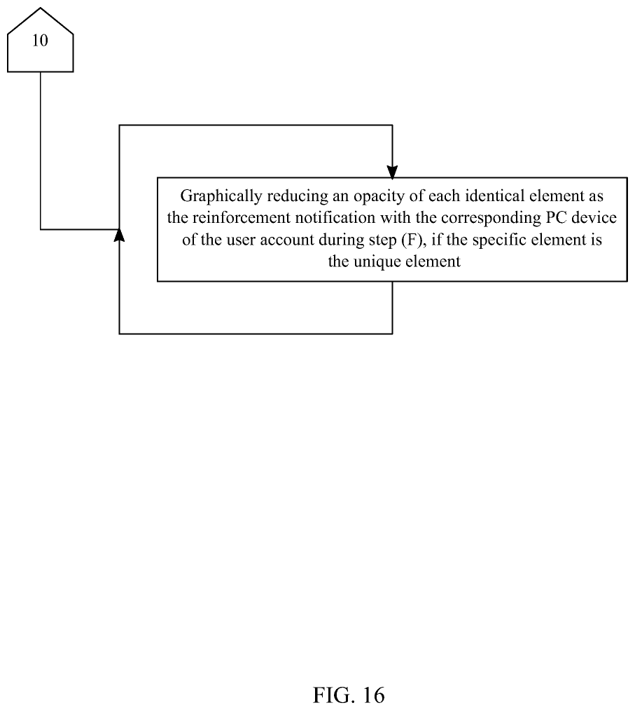

Similar to when the user improperly completes the specific task, the system of the present invention provides feedback to the user in the form of the reinforcement notification when the user properly completes the specific task. As can be seen in FIGS. 4 and 14, if the specific element selected by the user account is the unique element, the unique element is graphically enlarged as the reinforcement notification with the corresponding PC device of the user account during Step F. In other words, when the user selects the unique element, the user properly completes the specific task, and the unique element is enlarged to reinforce that the unique element was the correct selection. In addition, as can be seen in FIGS. 4 and 15, if the specific element selected by the user account is the unique element, the unique element is graphically bordered as the reinforcement notification with the corresponding PC device of the user account during Step F. By enlarging and highlighting the unique element when the user selects the unique element, the user is provided with positive reinforcement that improves the progression of the user's rehabilitation. Furthermore, as can be seen in FIGS. 4 and 16, if the specific element selected by the user account is the unique element, an opacity of each identical element is graphically reduced as the reinforcement notification with the corresponding PC device of the user account during Step F. By visually fading the plurality of identical elements, the user is further indicated that the selected specific element is the unique element, and that the other visual elements were the plurality of identical elements.

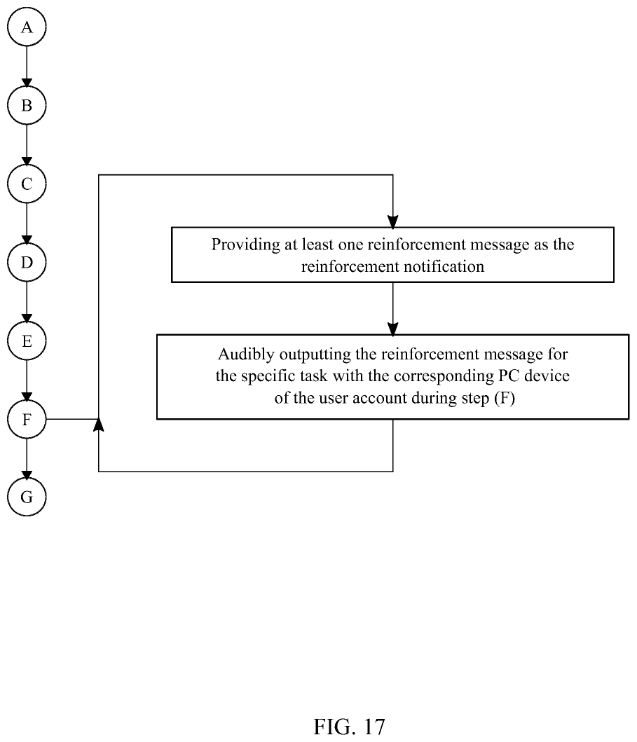

As can be seen in FIG. 17, to provide additional feedback to the user after properly completing the specific task, the system of the present invention further provides at least one reinforcement message as the reinforcement notification. The reinforcement message provides additional reinforcement information after the user has properly completed the specific task in addition to the specific hint and other reinforcement features. For example, the reinforcement message can provide audible information regarding the correct selection and how the unique element differentiated from the identical elements. The subprocess of providing a reinforcement message includes the step of audibly outputting the reinforcement message for the specific task with the corresponding PC device of the user account during Step F. This way, the user is provided with enough reinforcement to know how and why the user has properly completed the specific task.

As previously discussed, the present invention accommodates the need of the user to provide long-term neuropsychological rehabilitation. This can be done by modifying different parameters of the system of the present invention. The basic parameters of the programs include, but are not limited to, number of difficulty levels, starting level, number of tasks per game, postponing of repeated tasks, number of images per object, number of text elements per object, default time to respond, etc. Specific difficult levels can utilize different parameters which include, but are not limited to, objects in the pool for the visual elements, number of elements displayed, number of rows, number of columns (number of columns is automatically adjusted according to number of elements, rows, etc.), number of element groups displayed, deviation of elements, task timer, etc. Furthermore, the system of the present invention can provide various rehabilitation programs including, but not limited to, a unique object program, a unique letter program, a food product program, a mismatched animal program, a different result program, and a unique symbol program.

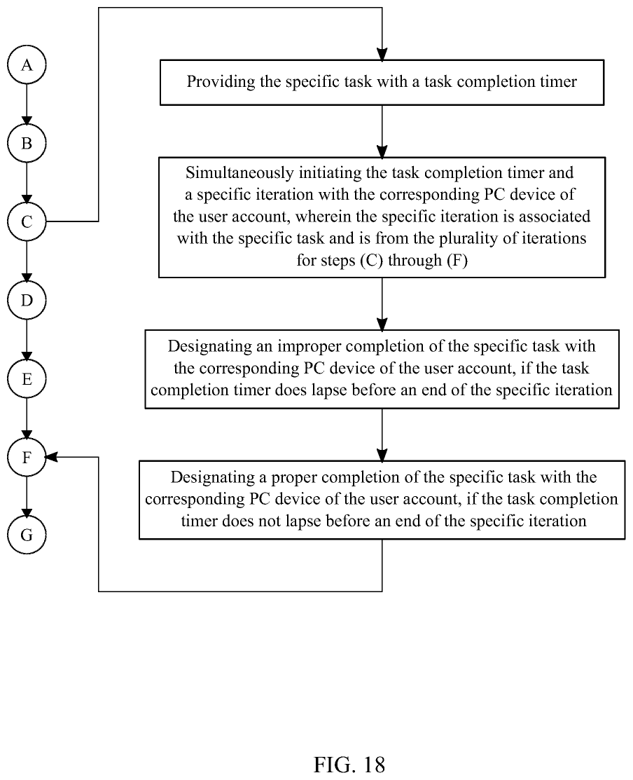

As can be seen in FIG. 18, to set the timer parameter of the specific task, the system of the present invention further provides the specific task with a task completion timer. The task completion timer limits the amount of time the user has to properly complete the specific task. The subprocess of providing a task completion timer with the specific task starts by simultaneously initiating the task completion timer and a specific iteration with the corresponding PC device of the user account. The specific iteration is associated with the specific task and is from the plurality of iterations for Steps C through F. In other words, every iteration for Steps C through F can have a different task completion timer which enables the change of difficulty for every specific task. If the task completion timer does lapse before an end of the specific iteration, an improper completion of the specific task is designated with the corresponding PC device of the user account. In other words, if the user fails to properly complete the specific task before the task completion timer runs out, the specific task is terminated, and the user cannot complete the specific task. Alternatively, if the task completion timer does not lapse before an end of the specific iteration, a proper completion of the specific task is designated with the corresponding PC device of the user account. In other words, a proper completion of the specific task only occurs if the specific task is properly completed within the task completion timer.

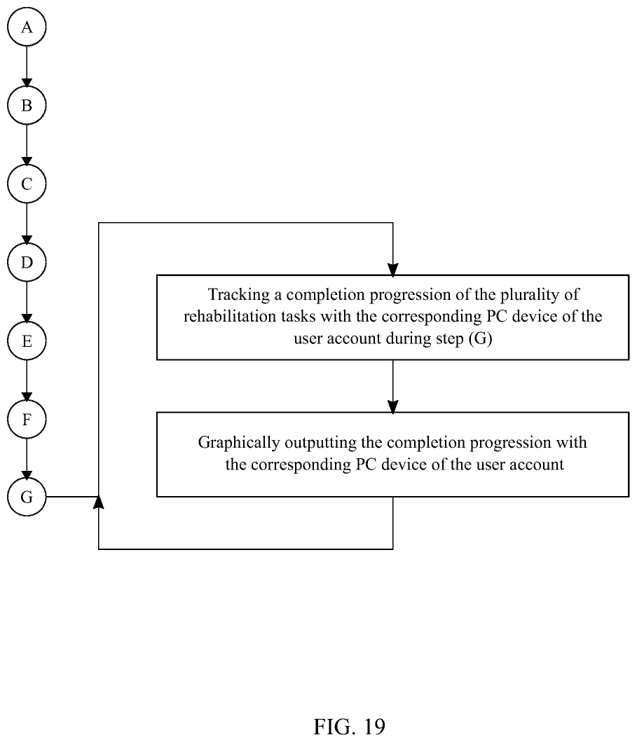

The treatment programs provided by the present invention can be tailored to match the user's needs. For example, the number of rehabilitation tasks can be modified to include enough rehabilitation tasks for the improvement of the user's cognitive functions. As can be seen in FIGS. 4 and 19, to help the user track the progress made while performing the treatment programs, the system of the present invention provides a way for the user to track progress. The subprocess of tracking the completion progress made by the user starts by tracking a completion progression of the plurality of rehabilitation tasks with the corresponding PC device of the user account during Step G. In other words, the system of the present invention tracks how much progress that the user has made by completing the plurality of rehabilitation tasks. Then, the completion progression is graphically outputted with the corresponding PC device of the user account. This way, the user is able to keep track of the completion progress to be encouraged to complete all the rehabilitation tasks.

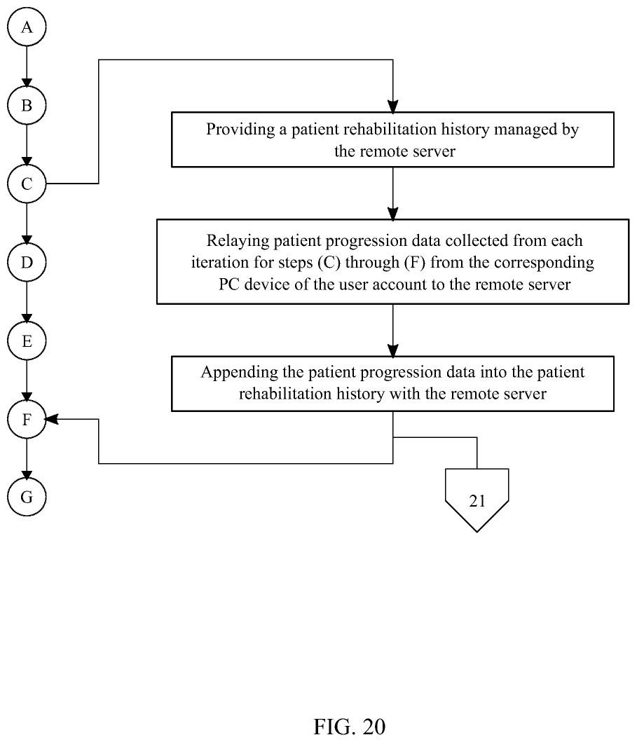

As can be seen in FIG. 20, to facilitate the customization of the treatment programs to the user, the system of the present invention provides a way for the remote server to track the completion progress of the user. To do so, the remote server further manages a patient rehabilitation history that logs the completion progress of the user. The subprocess of generating the patient rehabilitation history starts by relaying patient progression data collected from each iteration for Steps C through F from the corresponding PC device of the user account to the remote server. The patient progression data includes information about the user's progress as the user completes several rehabilitation tasks. For example, the success rate of completion, types of tasks the user is able to complete, types of tasks the user fails to complete, etc. This way, the whole progression is logged by the patient rehabilitation history. Then, the patient progression data is appended into the patient rehabilitation history with the remote server to ensure that all progress is logged into the patient rehabilitation history.

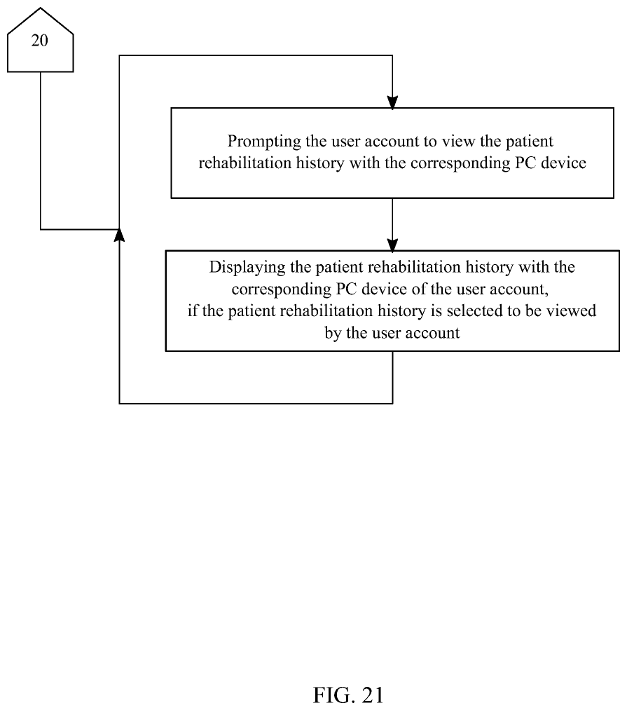

The patient rehabilitation history is preferably accessible to the user and to authorized users. As can be seen in FIG. 21, the subprocess of enabling the user access to the patient rehabilitation history starts by prompting the user account to view the patient rehabilitation history with the corresponding PC device. The user and/or the user's caretakers are able to view the patient rehabilitation history by using the corresponding PC device to have a better idea of the overall user's progress. Then, the patient rehabilitation history is displayed with the corresponding PC device of the user account, if the patient rehabilitation history is selected to be viewed by the user account. A similar process can be authorized to be done by other authorized users, such as medical professionals associated with the health and rehabilitation of the user.

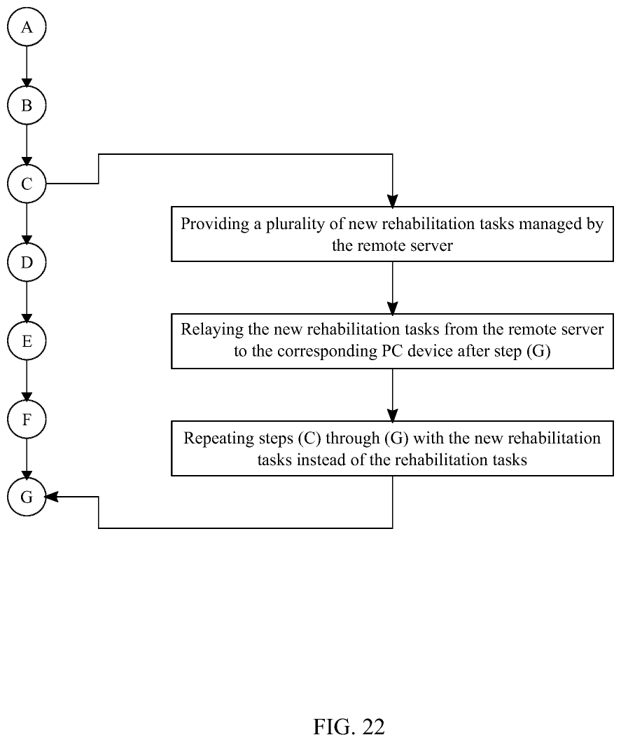

The patient rehabilitation history can be used to modify the plurality of rehabilitation tasks to match the rehabilitation progress of the user. As can be seen in FIG. 22, to do so, the remote server further manages a plurality of new rehabilitation tasks which are generated to match the rehabilitation progress of the user. The subprocess of providing a plurality of new rehabilitation tasks starts by relaying the new rehabilitation tasks from the remote server to the corresponding PC device after Step G. Once the user account receives the new rehabilitation task, Steps C through B are repeated with the new rehabilitation tasks instead of the rehabilitation tasks. In other words, the user continues to work on the rehabilitation tasks and the parameters of new rehabilitation tasks are modified to match the user's rehabilitation progress. Thus, the user is able to receive neuropsychological rehabilitation. The present invention gives users to professional, individual care so that the users can rehabilitation at their cognitive functions on a daily basis. Because of the adaptive system of the present invention, the users can work independently or with minimal assistance. By using the present invention, the users are able to understand things faster, make decisions with less hesitation, and have greater confidence. The users are expected to have increased cognitive functioning which is a vital part of the quality of life.

Although the invention has been explained in relation to its preferred embodiment, it is to be understood that many other possible modifications and variations can be made without departing from the spirit and scope of the invention.

What is claimed is:

1. A method of facilitating digital therapy for long-term neuropsychological rehabilitation, the method comprising the steps of:
(A) providing at least one user account and a plurality of rehabilitation tasks managed by at least one remote server, wherein the user account is associated with a corresponding personal computing (PC) device;
(B) prompting the user account to initiate the plurality of rehabilitation tasks with the corresponding PC device;
(C) prompting the user account to complete a specific task with the corresponding PC device, if the plurality of rehabilitation tasks is selected to be initiated by the user account;
(D) outputting at least one specific hint for the specific task with the corresponding PC device of the user account, if the specific task is improperly completed by the user account;
(E) repeating step (C), if the specific hint is outputted by the corresponding PC device, and until the specific task is properly completed by the user account;
(F) outputting at least one reinforcement notification for the specific task with the corresponding PC device of the user account, if the specific task is properly completed by the user account; and
(G) executing a plurality of iterations for steps (C) through (F), until each rehabilitation task is properly completed by the user account, wherein the specific task of each iteration for steps (C) through (F) is associated with a corresponding task from the plurality of rehabilitation tasks.

2. The method as claimed in claim 1 comprising the steps of:
providing the specific task with at least one task instruction; and
outputting the task instruction for the specific task with the corresponding PC device before step (C).

3. The method as claimed in claim 1 comprising the steps of:
providing the specific task with a plurality of visual elements, wherein the visual elements include a unique element and a plurality of identical elements;
displaying the unique element amongst the identical elements in a specific graphic distribution with the corresponding PC device of the user account during step (C);
prompting the user account to select a specific element from the plurality of visual elements with the corresponding PC device during step (C);
designating an improper completion of the specific task with the corresponding PC device of the user account, if the specific element is not the unique element; and
designating a proper completion of the specific task with the corresponding PC device of the user account, if the specific element is the unique element.

4. The method as claimed in claim 3 comprising the step of:
graphically reducing an opacity of the specific element as the specific hint with the corresponding PC device of the user account during step (D), if the specific element is not the unique element.

5. The method as claimed in claim 3 comprising the step of:
graphically removing the specific element as the specific hint with the corresponding PC device of the user account during step (D), if the specific element is not the unique element.

6. The method as claimed in claim 3 comprising the steps of:
further providing the specific task with a plurality of new visual elements, wherein the new visual elements include a new unique element and a plurality of new identical elements; and
repeating steps (C) through (F) with the new visual elements, the new unique element, and the new identical elements instead of the visual elements, the unique element, and the identical elements, if the specific element is not the unique element.

7. The method as claimed in claim 3 comprising the step of:
graphically enlarging the unique element as the reinforcement notification with the corresponding PC device of the user account during step (F), if the specific element is the unique element.

8. The method as claimed in claim 3 comprising the step of:
graphically bordering the unique element as the reinforcement notification with the corresponding PC device of the user account during step (F), if the specific element is the unique element.

9. The method as claimed in claim 3 comprising the step of:
graphically reducing an opacity of each identical element as the reinforcement notification with the corresponding PC device of the user account during step (F), if the specific element is the unique element.

10. The method as claimed in claim 1 comprising the steps of:
providing at least one reinforcement message as the reinforcement notification; and
audibly outputting the reinforcement message for the specific task with the corresponding PC device of the user account during step (F).

11. The method as claimed in claim 1 comprising the steps of:
providing the specific task with a task completion timer;
simultaneously initiating the task completion timer and a specific iteration with the corresponding PC device of the user account, wherein the specific iteration is associated with the specific task and is from the plurality of iterations for steps (C) through (F);
designating an improper completion of the specific task with the corresponding PC device of the user account, if the task completion timer does lapse before an end of the specific iteration; and
designating a proper completion of the specific task with the corresponding PC device of the user account, if the task completion timer does not lapse before an end of the specific iteration.

12. The method as claimed in claim 1 comprising the steps of:
tracking a completion progression of the plurality of rehabilitation tasks with the corresponding PC device of the user account during step (G); and
graphically outputting the completion progression with the corresponding PC device of the user account.

13. The method as claimed in claim 1 comprising the steps of:
providing a patient rehabilitation history managed by the remote server;
relaying patient progression data collected from each iteration for steps (C) through (F) from the corresponding PC device of the user account to the remote server; and
appending the patient progression data into the patient rehabilitation history with the remote server.

14. The method as claimed in claim 13 comprising the steps of:
prompting the user account to view the patient rehabilitation history with the corresponding PC device; and
displaying the patient rehabilitation history with the corresponding PC device of the user account, if the patient rehabilitation history is selected to be viewed by the user account.

15. The method as claimed in claim 1 comprising the steps of:
providing a plurality of new rehabilitation tasks managed by the remote server;
relaying the new rehabilitation tasks from the remote server to the corresponding PC device after step (G); and
repeating steps (C) through (G) with the new rehabilitation tasks instead of the rehabilitation tasks.
